(19) United States
(12) Patent Application Publication
(10) Pub. No.: US 2022/0008310 A1
(43) Pub. Date: Jan. 13, 2022

(54) COMPOSITION OF ACTIVE INGREDIENTS FOR CARING FOR AND MODIFIYING HUMAN HAIR

(71) Applicant: Henkel AG & Co. KGaA, Duesseldorf (DE)

(72) Inventors: RENE KROHN, Norderstedt (DE); ERIK SCHULZE ZUR WIESCHE, Bielefeld (DE)

(73) Assignee: Henkel AG & Co. KGaA, Duesseldorf (DE)

(21) Appl. No.: 17/289,581

(22) PCT Filed: Oct. 31, 2019
(86) PCT No.: PCT/EP2019/079774

§ 371 Date: Apr. 28, 2021

(30) Foreign Application Priority Data
Oct. 31, 2018 (DE) 10 2018 127 278.2

(51) Int. Cl. A61K 8/58; A61K 8/73; A61Q 5/06; A61Q 5/12

(52) U.S. Cl. CPC A61K 8/585; A61K 8/736; A61K 2800/5922; A61Q 5/12; A61K 2800/95; A61Q 5/06

(57) ABSTRACT

The present disclosure relates to an active ingredient composition for the care of human hair. In particular, the present disclosure relates to a cosmetic composition for treating a keratinous material comprising a) at least one organic silicon compound and b) at least one cationic polymer, the cosmetic composition being particularly suitable for caring for damaged hair.

### CROSS-REFERENCE TO RELATED APPLICATION

This application is a U.S. National-Stage entry under 35 U.S.C. § 371 based on International Application No. PCT/EP2019/079774, filed Oct. 31, 2019, which was published under PCT Article 21(2) and which claims priority to German Application No. 10 2018 127 278.2, filed Oct. 31, 2018, which are all hereby incorporated in their entirety by reference.

### TECHNICAL FIELD

The present disclosure relates to cosmetic compositions for treating a keratinous material, the composition comprising as a first component an organic silicon compound and as a second component at least one cationic polymer, and to the use of the cosmetic composition.

### BACKGROUND

The external exposure of hair to chemicals from a variety of different sources poses challenges for the development of cosmetic care products. Air and water impurities have a detrimental effect on skin and hair. Major air pollutants include polycyclic aromatic hydrocarbons, volatile organic compounds, nitrogen oxides (NOx), particulate matter, and cigarette smoke. The effect of various air pollutants can be enhanced in the presence of other air pollutants and when exposed to UV radiation.

It is known that the toxicity of gaseous pollutants in the air, such as sulfur dioxide, ozone, and nitrogen oxides, is related to their initiator activity for free radicals, which cause damage to living organisms. Free radicals are metabolic products that also occur naturally in the body. In large quantities, free radicals can promote irritation and inflammation and accelerate the process of aging. In this case, the term “oxidative damage” is used. Free radicals can also cause hair damage, which is visible, for example, as a reduction in shine as well as grip and/or fading of hair color.

Furthermore, often changing consumer demands for a certain hair texture are associated with recurring chemical exposure of the hair. For example, hair coloring stresses the hair, due to which a special, intensive care may be necessary.

In the prior art, organosilicon compounds from the group of silanes comprising at least one hydroxy group and/or hydrolysable group are described. Due to the presence of the hydroxy groups and/or hydrolysable groups, the silanes are reactive substances that hydrolyze or oligomerize or polymerize in the presence of water. The oligomerization or polymerization of the silanes initiated by the presence of the water, when applied to a keratinous material, ultimately leads to the formation of a film that can exert a protective effect.

### BRIEF SUMMARY

Cosmetic compositions for treating keratinous materials, and methods of using the same, are provided. In an exemplary embodiment, a cosmetic composition for treating keratinous materials includes at least one organic silicon compound and at least one cationic polymer.

A method of using a cosmetic composition for treating keratinous materials is provided in another embodiment. The method includes applying the cosmetic composition to the keratinous materials, where the cosmetic composition includes an organic silicon compound and a cationic polymer.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The task underlying the present disclosure is to provide a product with an improved care and/or protection effect. In particular, the present disclosure was based on the task of providing a cosmetic agent which, after a hair treatment, enables a caring after-treatment which provides special care to stressed hair.

This task is solved by a cosmetic agent for the treatment of a keratinous material, comprising

a) at least one organic silicon compound and
b) at least one cationic polymer.

By a keratinous material is meant hair, the skin, and the nails (such as fingernails and/or toenails). Wool, furs, and feathers also fall under the definition of keratinous material.

Preferably, a keratinous material is understood to mean human hair, human skin, and human nails, in particular fingernails and toenails. Very preferably, keratinous material is understood to mean human hair, in particular head and/or beard hair.

As a first ingredient essential to the composition described herein, the cosmetic composition for treating a keratinous material contains at least one organic silicon compound. Preferred organic silicon compounds are selected from silanes having one, two or three silicon atoms, wherein the organic silicon compound comprises one or more hydroxyl groups and/or hydrolysable groups per molecule.

Organic silicon compounds, alternatively called organosilicon compounds, are compounds which either have a direct silicon-carbon bond (Si—C) or in which the carbon is bonded to the silicon atom via an oxygen, nitrogen, or sulfur atom. The organic silicon compounds are compounds containing one to three silicon atoms. Organic silicon compounds preferably contain one or two silicon atoms.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

The agent for treating a keratinous material contains at least one organic silicon compound preferably selected from silanes having one, two or three silicon atoms, wherein the organic silicon compound comprises one or more hydroxyl groups or hydrolysable groups per molecule.

In a most preferred embodiment, the agent for treating a keratinous material comprises at least one organic silicon compound selected from silanes having one, two or three silicon atoms, wherein the organic silicon compound further comprises one or more basic groups and one or more hydroxyl groups or hydrolysable groups per molecule.

This basic group can be, for example, an amino group, an alkylamino group or a dialkylamino group, which is preferably connected to a silicon atom via a linker. The basic group is preferably an amino group, a C₁-C₆ alkylamino group or a di(C₁-C₆)alkylamino group.

The hydrolysable group(s) is (are) preferably a C₁-C₆ alkoxy group, especially an ethoxy group or a methoxy group. It is preferred when the hydrolysable group is directly bonded to the silicon atom. For example, if the hydrolysable group is an ethoxy group, the organic silicon compound preferably contains a structural unit R′R″R′″Si—O—CH₂—CH₃. The residues R′, R″ and R′″ represent the three remaining free valences of the silicon atom.

Particularly good results were obtained when the agent for treating a keratinous material contains at least one organic silicon compound of formula (I) and/or (II).

The compounds of formulae (I) and (II) are organic silicon compounds selected from silanes having one, two or three silicon atoms, the organic silicon compound comprising one or more hydroxyl groups and/or hydrolysable groups per molecule.

In another very particularly preferred embodiment, the agent for treating a keratinous material comprises at least one organic silicon compound of formula (I) and/or (II),

R₁R₂N-L-Si(OR₃)ₐ(R₄)_{b} (I),
where

R₁, R₂ both represent a hydrogen atom,

L represents a linear, divalent C₁-C₆-alkylene group, preferably a propylene group (—CH₂—CH₂—CH₂—) or an ethylene group (—CH₂—CH₂—),

R₃, R₄ independently represent a methyl group or an ethyl group,

a stands for the number 3 and

b stands for the number 0.

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
where

R5, R5′, R5″ independently represent a hydrogen atom or a C₁-C₆ alkyl group,
R6, R6′ and R6″ independently represent a C₁-C₆ alkyl group,
A, A′, A″, A′″ and A″″ independently represent a linear or branched C₁-C₂₀ divalent alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d}″(OR₅″)_{c}″ (III),

c, stands for an integer from 1 to 3,

d stands for the integer 3-c,

c′ stands for an integer from 1 to 3,

d′ stands for the integer 3-c′,

c″ stands for an integer from 1 to 3,

d″ stands for the integer 3-c″,

e stands for 0 or 1,

f stands for 0 or 1,

g stands for 0 or 1,

h stands for 0 or 1,

provided that at least one of e, f, g, and h is different from 0.

The substituents R₁, R₂, R₃, R₄, R₅, R₅′, R₅″, R₆, R₆′, R₆″, R₇, R₈, L, A, A′, A″, A′″ and A″″ in the compounds of formula (I) and (II) are explained below as examples: Examples of a C₁-C₆ alkyl group are the groups methyl, ethyl, propyl, isopropyl, n-butyl, s-butyl, and t-butyl, n-pentyl and n-hexyl. Propyl, ethyl, and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, and the 3-aminopropyl group. The 2-aminoethyl group is particularly preferred. Examples of a linear divalent C₁-C₂₀ alkylene group include the methylene group (—CH₂—), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly preferred. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L- which stands for a linear or branched, divalent C₁-C₂₀ alkylene group.

Preferably, -L- represents a linear, divalent C₁-C₂₀ alkylene group. Further preferably, -L- represents a linear divalent C₁-C₆ alkylene group. Particularly preferred -L stands for a methylene group (—CH₂—), an ethylene group (—CH₂—CH₂—), a propylene group (—CH₂—CH₂—CH₂—) or a butylene (—CH₂—CH₂—CH₂—CH₂—). L very particularly stands for a propylene group (—CH₂—CH₂—CH₂—).

The organic silicon compounds of formula (I)

R₁R₂N-L-Si(OR₃)ₐ(R₄)_{b} (I),

carry the silicon-containing grouping —Si(OR₃)ₐ(R₄)_{b} at one end.

In the terminal structural unit —Si(OR₃)ₐ(R₄)_{b}, R₃ is hydrogen or a C₁-C₆ alkyl group, and R₄ is a C₁-C₆ alkyl group. R₃ and R₄ particularly preferably independently of each other represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

The best protection against the negative effects of water and/or air pollution (“anti-pollution” effect) and the best care of stressed hair could be obtained if the agent for treating a keratinous material contains at least one organic silicon compound of formula (I) in which the radicals R₃, R₄ independently represent a methyl group or an ethyl group.

Particularly well-suited organic silicon compounds of formula (I) are

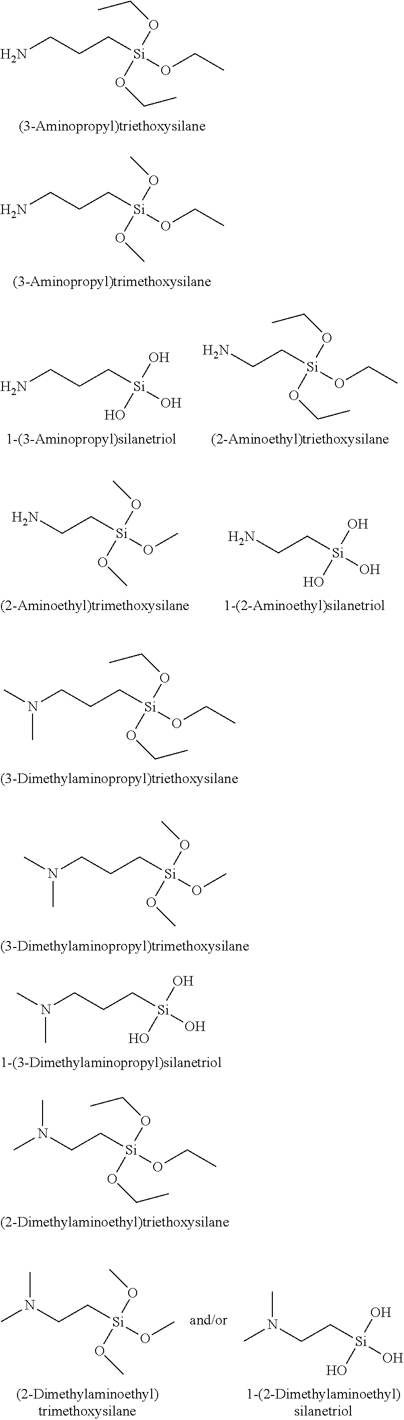

The organic silicon compound of formula (I) is commercially available. (3-aminopropyl)trimethoxysilane, for example, can be purchased from Sigma-Aldrich®. (3-Aminopropyl)triethoxysilane is also commercially available from Sigma-Aldrich®.

In another embodiment, the composition for treating a keratinous material comprises at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II).

The organosilicon compounds of formula (II) each bear at their two ends the silicon-containing groupings (R₅O)_{c}(R₆)_{d}Si— and —Si(R₆′)_{a′}(OR₅′)_{c′}-.

In the central part of the molecule of formula (II) there are the groups -(A)ₑ- and —[NR₇-(A′)]_{f}- and —[O-(A″)]_{g}- and —[NR₈-(A′″)]ₕ-. Here, each of the radicals e, f, g, and h can independently of one another stand for the number 0 or 1, with the proviso that at least one of the radicals e, f, g, and h is different from 0. In other words, an organic silicon compound of formula (II) contains at least one grouping selected from the group of -(A)- and —[NR₇-(A′)]- and —[O-(A″)]- and —[NR₈-(A′″)]-.

In the two terminal structural units (R₅O)_{c}(R₆)_{d}Si— and —Si(R₆′)_{d′}(OR₅′)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here d stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

An extremely high anti-pollution effect of the agent for the treatment of a keratinous material could be obtained when the residues c and c′ both stand for the number 3. In this case d and d′ both stand for the number 0.

In another preferred embodiment, the agent for treating a keratinous material comprises at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where

R5 and R5′ independently represent a methyl group or an ethyl group,

c and c′ both stand for the number 3 and

d and d′ both stand for the number 0.

When c and c′ both represent the number 3 and d and d′ both represent the number 0, the organic silicon compounds correspond to formula (IIa)

(R₅O)₃Si-(A)_{c}-[NR₇-(A′)]_{f}-[O-(A″)]_{g}—[NR₈-(A′″)]ₕ-Si(OR₅′)₃ (IIa).

The radicals e, f, g, and h can independently stand for the number 0 or 1, whereby at least one radical from e, f, g, and h is different from zero. The abbreviations e, f, g, and h thus define which of the groupings -(A)_{c}- and —[NR7-(A′)]_{f}- and —[O-(A″)]_{g}- and —[NR8-(A′″)]ₕ- are in the middle part of the organic silicon compound of formula (II).

In this context, the presence of certain groupings has proven to be particularly beneficial in terms of increasing the “anti-pollution” effect. Particularly good results were obtained when at least two of the residues e, f, g, and h stand for the number 1. Especially preferred e and f both stand for the number 1. Furthermore, g and h both stand for the number 0.

When e and f are both 1 and g and h are both 0, the organic silicon compounds are represented by the formula (IIb)

(R₅O)_{c}(R₆)_{d}Si-(A)-[NR₇-(A′)]—Si(R₆′)_{d′}(OR₅′)_{c′} (IIb).

The radicals A, A′, A″, A′″ and A″″ independently represent a linear or branched divalent C₁-C₂₀ alkylene group. Preferably the radicals A, A′, A″, A′″ and A″″ independently of one another represent a linear, divalent C₁-C₂₀ alkylene group. Further preferably the radicals A, A′, A″, A′″ and A″″ independently represent a linear divalent C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

When the radical f represents the number 1, the organic silicon compound of formula (II) contains a structural grouping —[NR₇-(A′)]-.

When the radical h represents the number 1, the organic silicon compound of formula (II) contains a structural grouping —[NR₈-(A″)]-.

Wherein R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

(A″″)—Si(R₆″)_{d}″(OR₅″)_{c}″ (III).

Very preferably, R₇ and R₈ independently represent a hydrogen atom, a methyl group, a 2-hydroxyethyl group, a 2-alkenyl group, a 2-aminoethyl group or a group of formula (III).

When the radical f represents the number 1 and the radical h represents the number 0, the organic silicon compound contains the grouping [NR₇-(A′)] but not the grouping —[NR₈-(A′″)]. If the radical R7 now stands for a grouping of the formula (III), the agent for treating a keratinous material contains an organic silicon compound with 3 reactive silane groups.

In another preferred embodiment, the agent for treating a keratinous material comprises at least one organic silicon compound of formula (II)

(R₅O)_{c}(R₆)_{d}Si-(A)ₑ-[NR₇-(A′)]_{f}-[O-(A″)]_{g}—[NR₈-(A′″)]ₕ-Si(R₆′)_{d′}(OR₅′)_{c′} (II),

where

e and f both stand for the number 1,

g and h both stand for the number 0,

A and A′ independently represent a linear, divalent C₁-C₆ alkylene group and

R7 represents a hydrogen atom, a methyl group, a 2-hydroxyethyl group, a 2-alkenyl group, a 2-aminoethyl group or a group of formula (III).

In another preferred embodiment, the composition for treating a keratinous material comprises at least one organic silicon compound of the formula (II), wherein

e and f both stand for the number 1,

g and h both stand for the number 0,

A and A′ independently of one another represent a methylene group(—CH₂—), an ethylene group (—CH₂—CH₂—) or a propylene group (—CH₂—CH₂—CH₂), and

R₇ represents a hydrogen atom, a methyl group, a 2-hydroxyethyl group, a 2-alkenyl group, a 2-aminoethyl group or a group of formula (III).

Organic silicon compounds of formula (II) that are well suited for solving the problem are;

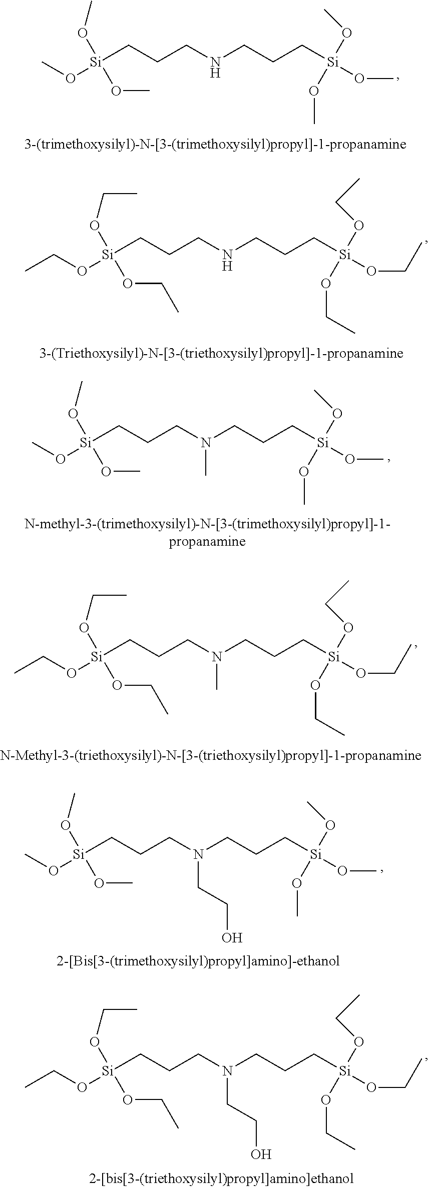

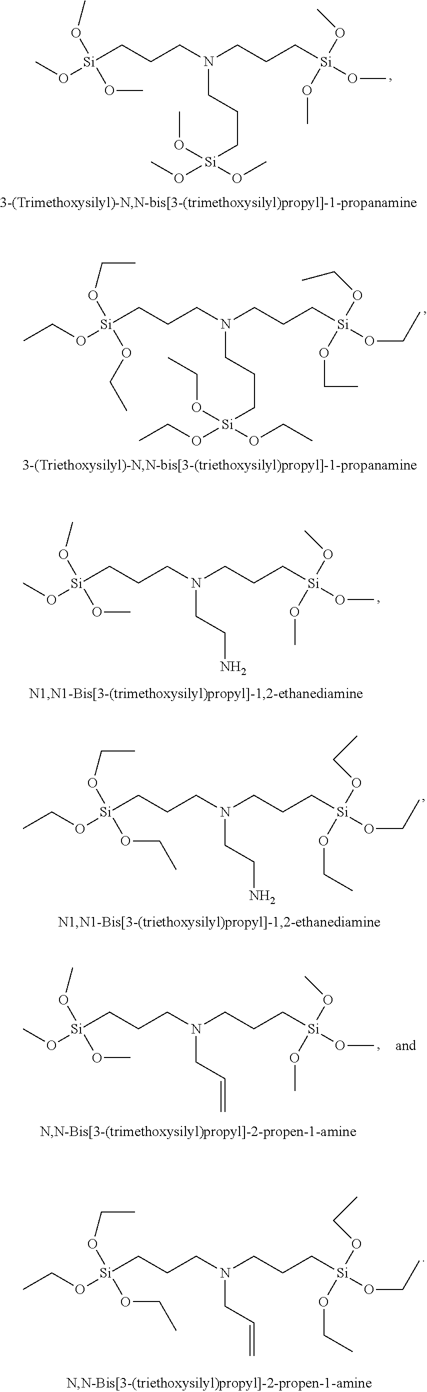

The organic silicon compounds of formula (II) are commercially available.

Bis(trimethoxysilylpropyl)amine with the CAS number 82985-35-1 can be purchased from Sigma-Aldrich®.

Bis[3-(triethoxysilyl)propyl]amine, also known as 3-(triethoxysilyl)-N-[3-(triethoxysilyl)propyl]-1-propanamine, with CAS number 13497-18-2 can be purchased, for example, from Sigma-Aldrich® or is commercially available under the product name Dynasylan® 1122 from Evonik®.

N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich® or Fluorochem®.

3-(triethoxysilyl)-N,N-bis[3-(triethoxysilyl)propyl]-1-propanamine with the CAS number 18784-74-2 can be purchased for example from Fluorochem® or Sigma-Aldrich®.

It has also been found to be advantageous when the agent for treating a keratinous material applied to the hair contains at least one organic silicon compound of formula (IV)

R₉Si(OR₁₀)ₖ(R₁₁)ₘ (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be referred to as silanes of the alkylalkoxysilane or alkylhydroxysilane type,

R₉Si(OR₁₀)ₖ(R₁₁)ₘ (IV),

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In a further preferred embodiment, the composition for treating a keratinous material contains, in addition to the organic silicon compound or compounds of formula (I), at least one further organic silicon compound of formula (IV)

R₉Si(OR₁₀)ₖ(R₁₁)ₘ (IV),

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In a likewise preferred embodiment, the composition for treating a keratinous material contains, in addition to the organic silicon compound or compounds of the formula (II), at least one further organic silicon compound of the formula (IV)

R₉Si(OR₁₀)ₖ(R₁₁)ₘ (IV),

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In another preferred embodiment, the composition for treating a keratinous material contains, in addition to the organic silicon compounds of the formula (I) and (II), at least one further organic silicon compound of the formula (IV)

R₉Si(OR₁₀)ₖ(R₁₁)ₘ (IV),

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In the organic silicon compounds of formula (IV), the radical R₉ represents a C₁-C₁₂ alkyl group. This C₁-C₁₂ alkyl group is saturated and can be linear or branched. Preferably R9 stands for a linear C₁-C₅ alkyl group. Preferably R₉ stands for a methyl group, an ethyl group, an n-propyl group, an n-butyl group, an n-pentyl group, an n-hexyl group, an n-octyl group or an n-dodecyl group. Particularly preferably, R₉ represents a methyl group, an ethyl group or an n-octyl group.

In the organic silicon compounds of formula (IV), the radical R₁₀ represents a hydrogen atom or a C₁-C₆ alkyl group. Particularly preferably, R10 represents a methyl group or an ethyl group.

In the organic silicon compounds of formula (IV), the radical R₁₁ represents a C₁-C₆ alkyl group. Particularly preferably, R₁₁ represents a methyl group or an ethyl group.

Furthermore, k stands for a whole number from 1 to 3, and m stands for the whole number 3-k. If k stands for the number 3, then m is equal to 0. If k stands for the number 2, then m is equal to 1. If k stands for the number 1, then m is equal to 2.

An extremely high “anti-pollution” effect could be obtained if the agent for treating a keratinous material contains at least one organic silicon compound of formula (IV) in which the radical k represents the number 3. In this case the residue m stands for the number 0.

Organic silicon compounds of the formula (IV) that are particularly suitable for solving the problem are

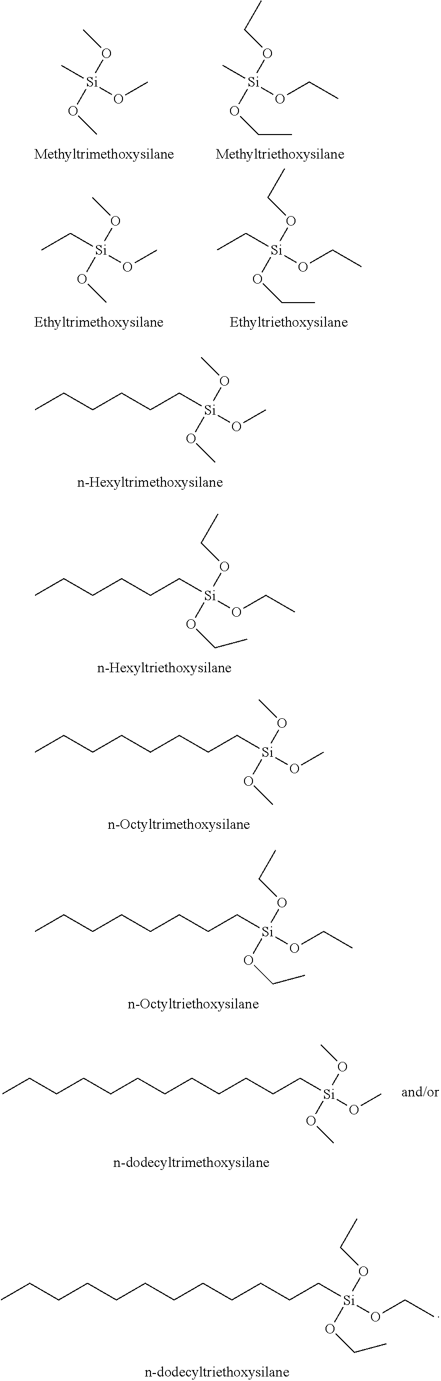

and propyltrimethoxysilane, propyltriethoxysilane, octadecyltrimethoxysilane and/or octadecyltriethoxysilane.

The organic silicon compounds described above are reactive compounds.

In this context, it has been found to be quite preferred if the agent contains as an organic silicon compound (3-aminopropyl)triethoxysilane, i.e., an aminopropyltriethoxysilane (AMEO), and/or 3-(triethoxysilyl)-N-[3-(triethoxysilyl)propyl]-1-propanamine, i.e., a bis(triethoxysilylpropyl)amine.

According to preferred embodiments of the present disclosure, the organic silicon compound of formula (I), in particular the (3-aminopropyl)triethoxysilane, is present in an amount of from about 0.01 to about 10% by weight, preferably from about 0.02 to about 8% by weight, more preferably from about 0.05 to about 6% by weight, most preferably from about 0.1 to about 4% by weight, based on the total weight of the cosmetic product, in the cosmetic product, and/or the organic silicon compound of the formula (II), in particular the 3-(triethoxysilyl)-N-[3-(triethoxysilyl)propyl]-1-propanamine, is present in an amount of from about 0.01 to about 10% by weight, preferably from about 0.02 to about 9% by weight, more preferably from about 0.05 to about 8% by weight, most preferably from about 0.1 to about 7%, by weight, based on the total weight of the cosmetic composition, in the cosmetic composition.

It was found that particularly stable and uniform films could be obtained on the keratinous material even when the agent contained two structurally different organic silicon compounds.

In a preferred embodiment, an exemplary agent comprises at least one organic silicone compound of formula (I) and at least one organic silicone compound of formula (IV).

In an explicitly very particularly preferred embodiment, an agent exemplified in that it contains at least one organic silicone compound of formula (I) selected from the group of (3-aminopropyl)triethoxysilane and (3-aminopropyl)trimethoxysilane, and additionally contains at least one organic silicone compound of formula (IV) selected from the group of methyltrimethoxysilane, methyltriethoxysilane, ethyltrimethoxysilane, ethyltriethoxysilane, propyltrimethoxysilane, propyltriethoxysilane, hexyltrimethoxysilane and hexyltriethoxysilane.

In another preferred embodiment, an agent is exemplified in that the agent contains, based on the total weight of the agent:

from about 0.5 to about 5% by weight of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane, (2-dimethylaminoethyl)trimethoxysilane, and (2-dimethylaminoethyl)triethoxysilane, and

from about 3.2 to about 10% by weight of at least one second organic silicon compound selected from the group of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octyldecyltrimethoxysilane and octyldecyltriethoxysilane.

Even the addition of small amounts of water leads to hydrolysis in organic silicon compounds with at least one hydrolysable group. The hydrolysis products and/or organic silicon compounds having at least one hydroxy group may react with each other in a condensation reaction. For this reason, both the organosilicon compounds having at least one hydrolysable group and their hydrolysis and/or condensation products may be present in the composition. When organosilicon compounds having at least one hydroxyl group are used, both the organic silicon compounds having at least one hydroxyl group and their condensation products may be present in the composition.

A condensation product is understood to be a product formed by the reaction of at least two organic silicon compounds each having at least one hydroxyl group or hydrolysable group per molecule with elimination of water and/or with elimination of an alkanol. The condensation products can be, for example, dimers, but also trimers or oligomers, with the condensation products being in equilibrium with the monomers. Depending on the amount of water used or consumed in the hydrolysis, the equilibrium shifts from monomeric organic silicon compounds to condensation product.

In the context of the present disclosure, figures in wt.-% are—unless otherwise stated—always based on the total weight of the cosmetic product.

As a second ingredient essential to the composition described herein, the cosmetic composition for treating a keratinous material contains, as a further component b), at least one cationic polymer. In the course of the work leading to the present disclosure, it has been found that, to achieve a particularly good care effect, it is especially advantageous if the organic silicon compounds, for example (3-aminopropyl)triethoxysilane, i. e. (3-aminopropyl)triethoxysilane, i.e., an aminopropyltriethoxysilane (AMEO), or for example 3-(triethoxysilyl)-N-[3-(triethoxysilyl)propyl]-1-propanamine, i.e., a bis(triethoxysilylpropyl)amine, is combined with at least one cationic polymer.

The term cationic polymer refers to a polymer that has a cationic formal charge. This may be located along the backbone of the polymer or may be present in a side group. The cationic formal charge can also result from protonation in an acidic environment. The term “polymer” has the ordinary meaning known to those skilled in the art.

It was found that the combination of the cationic polymer on the one hand and the organosilicon compound, aminopropyltriethoxysilane (AMEO) and/or bis(triethoxysilylpropyl)amine, on the other hand, is particularly powerful in terms of a conditioning aftertreatment. The combination of a cationic polymer and said silanes occupying the hair surface is particularly effective in solving the problem underlying the present disclosure. The hair surface is hydrophobized, reducing frizz. The hair becomes softer, especially in the case of a previously exerted high chemical stress.

According to preferred embodiments of the present disclosure, the cationic polymer is composed of monomer units having a cationic formal charge, preferably having an amine group, an amide group or a quaternized nitrogen group, wherein the amount of monomer units having a cationic formal charge exceeds the amount of monomer units having a formal charge other than the cationic formal charge. This means that zwitterionic polymers may also be present in the cosmetic product, but in that case the number of groups with a cationic formal charge exceeds the number of groups with an anionic formal charge.

According to preferred embodiments of the present disclsoure, the cationic polymer is selected from the group of Acrylamidopropyltrimonium Chloride/Acrylamide Copolymer, Acrylamidopropyltrimonium Chloride/Acrylates Copolymer, Acrylates/Octylacrylamide/Diphenyl Amodimethicone Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Aminoethylpropanediol-Acrylates/Acrylamide Copolymer, Diethylene Glycolamine/Epichlorohydrin/Piperazine Copolymer, Polyacrylamide, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polyquaternium-67, Polyquaternium-72, Vinylamine/Vinyl Alcohol Copolymer, quaternized polyvinyl alcohol, cationic alkyl polyglycosides and Vinyl Caprolactam/VP/Dimethylaminoethyl Methacrylate Copolymer. Furthermore, the amidoamine and/or cationized amidoamine, cationized honey, cationic guar derivatives or poly(methacryloyloxyethyltrimethylammonium compounds) are preferred.

The cationic polymer chitosan is particularly preferred.

According to a preferred embodiment, component b) comprising the cationic polymer is used with another skin moisturizing agent in the cosmetic composition. The further skin moisturizing agent is selected from the group of glycerin, urea, hyaluronic acid, silanol esters of hyaluronic acid, panthenol, taurine, ceramides, phytosterols, aloe vera extracts, creatine, creatinine, sodium hyaluronate, polysaccharides, bio saccharides gum-1, cucumber extracts, butylene glycol, propylene glycol, methyl propanediol, ethylhexyl glycerol, sorbitol, amino acids, glycine, glycine soy, histidine, tyrosine or tryptophan being particularly preferred amino acids, amino acid derivatives, natural betaine compounds, lactic acid, lactates, in particular sodium lactate, and/or ethylhexyloxyglycerol. In particular, the selection of these additional skin moisturizers increases the caring character of the cosmetic product. Among these skin moisturizers may be cationic polymers. These are then present in addition to the cationic polymers used as contemplated herein.

According to a preferred embodiment of the present disclosure, component b) is present in a total amount in the cosmetic composition which is from about 0.1 to about 10% by weight, preferably from about 0.5 to about 8% by weight, more preferably from about 1 to about 6% by weight.

In particular, the features for treating a keratinous material may comprise a composition for cleaning a keratinous material, a composition for maintaining a keratinous material, a composition for maintaining and cleaning a keratinous material, and/or a composition for temporarily reshaping a keratinous material.

In the following, further ingredients of the hair treatment products are described, which may be contained in the products in addition to the previously described mandatory ingredients.

It may be preferred that the agent for treating a keratinous material further comprises from about 0.001 to about 20% by weight of at least one quaternary compound that is not a polymeric compound. This applies to agents for the care of a keratinous material and agents for the care and cleaning of a keratinous material.

It is preferred that the at least one quaternary compound is selected from at least one of the groups of:

i) the monoalkylquats and/or
ii) the esterquats and/or
iii) the quaternary imidazolines of formula (Tkat2),

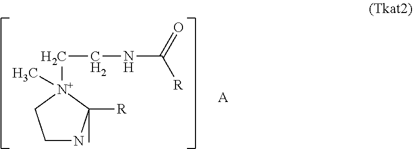

in which the radicals R independently of one another each represent a saturated or unsaturated, linear, or branched hydrocarbon radical having a chain length of from about 8 to about 30 carbon atoms and A represents a physiologically tolerated anion, and mixtures thereof.

It may be preferred that the agent for treating a keratinous material further comprises a firming compound, preferably selected from the group of waxes, synthetic polymers, and mixtures thereof.

To meet the different requirements for agents for the treatment of a keratinous material in the form of an agent for the temporary reshaping of a keratinous material (=styling agent), many synthetic polymers have already been developed as strengthening compounds, which can be used in the agent for the treatment of a keratinous material. Alternatively, or complementarily, waxes are used as strengthening compounds. Ideally, the polymers and/or waxes, when applied to the keratinous material, result in a polymer film or sheet that, on the one hand, gives the hairstyle a strong hold, but, on the other hand, is sufficiently flexible not to break when stressed.

Other anionic or nonionic polymers may be present in the cosmetic product. If among the following polymers are cationic polymers or polymers with cationizable groups, these may additionally be present in the cosmetic composition in addition to the cationic polymers used as contemplated herein.

Suitable synthetic polymers include, for example, polymers with the following INCI designations: Acrylamide/Ammonium Acrylate Copolymer, Acrylamides/DMAPA Acrylates/Methoxy PEG Methacrylate Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Stearyl Acrylate/Ethylamine Oxide Methacrylate Copolymer, Acrylates/VA Copolymer, Acrylates/Hydroxyesters Acrylates Copolymer, Acrylates/VP Copolymer, Allyl Stearate/VA Copolymer, Aminoethylacrylate Phosphate/Acrylates Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer. Cellulose ethers such as hydroxypropyl cellulose, hydroxyethyl cellulose and methyl hydroxypropyl cellulose are also suitable.

Also, homopolyacrylic acid (INCI: Carbomer), which is commercially available under the name Carbopol® in various forms, is suitable as a firming compound.

Preferably, the firming compound comprises a vinylpyrrolidone-containing polymer. Particularly preferably, the firming compound comprises a polymer selected from the group of polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), and mixtures thereof.

If cationic polymers are present among the strengthening compounds disclosed herein, they are used in addition to the cationic polymers used as contemplated herein.

Another preferred firming compound is octylacrylamide/acrylates/butylaminoethyl methacrylate copolymer (INCI), which is marketed under the name “Amphomer®” by Akzo Nobel®.

Accordingly, it is particularly preferred that the firming compound comprises a synthetic polymer selected from the group of polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), octylacrylamide/acrylates/butylaminoethyl methacrylate copolymer (INCI), and mixtures thereof.

According to further preferred embodiments of the present disclosure, the cosmetic composition comprises at least one cationic surfactant as component d). This is particularly preferably a cationic surfactant of formula (V),

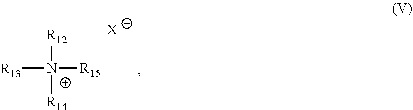

wherein
R₁₂, R₁₃, R₁₄ independently represent a C1-C6 alkyl group, a C2-C6 alkenyl group or a C2-C6 hydroxyalkyl group,
R₁₅ is a C8-C28 alkyl group, preferably a C10-C22 alkyl group, and
X− represents a physiologically compatible anion,
and/or the cosmetic composition preferably comprises at least one cationic surfactant of the formula (VI),

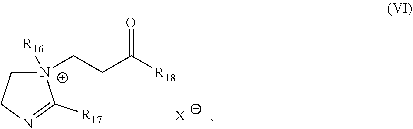

wherein
R₁₆ represents a C1-C6 alkyl group
R₁₇, R₁₈ independently represent a C7-C27 alkyl group, preferably a C10-C22 alkyl group, and
X− represents a physiologically compatible anion,
and/or the cosmetic composition preferably comprises at least one cationic surfactant of the formula (VII),

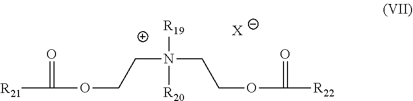

wherein
R₁₉, R₂₀ independently represent a C1-C6 alkyl group or a C2-C6 hydroxyalkyl group,
R₂₁, R₂₂ independently represent a C7-C27 alkyl group, preferably a C10-C22 alkyl group, and
X− represents a physiologically compatible anion,
and/or the cosmetic composition preferably comprises at least one cationic surfactant of the formula (VIII),

NR₂₃R₂₄R₂₅ (VIII)

wherein
R₂₃, R₂₄ independently represent a C1-C6 alkyl group, a C2-C6 alkenyl group or a C2-C6 hydroxyalkyl group, and
R₂₅ represents a C8-C28 alkyl group, preferably a C10-C22 alkyl group,

The cationic surfactants of formula (VIII) are amine derivatives, so-called pseudoquats. The organic radicals R₂₃, R₂₄ and R₂₅ are directly bonded to the nitrogen atom. In the acidic pH range, these are cationized, i.e., the nitrogen atom is then protonated. The physiologically compatible counterions are suitable as counterions. Steamidopropyl dimethylamine is particularly preferred among the cationic surfactants of formula (VIII).

According to a preferred embodiment of the present disclosure, the amount of cationic surfactant is from about 0.1 to about 30% by weight, preferably from about 0.5 to about 20% by weight, more preferably from about 1 to about 10% by weight, based on the total weight of the cosmetic composition.

According to a preferred embodiment of the present disclosure, the cationic surfactant comprises a hydrophobic head group with a cationic charge and one or two hydrophobic end portions, wherein the hydrophobic end portion(s) represent straight-chain or branched, saturated or mono- or polyunsaturated alkyl groups, preferably having a chain length of C6 to C30, more preferably C8 to C26, particularly preferably C10 to C22. According to another preferred embodiment, the cationic surfactant has an ester function, an ether function, a ketone function, an alcohol function, or an amide function.

According to a preferred embodiment of the present disclsoure, the cosmetic composition further comprises a nonionic surfactant. This preferably comprises a nonionic surfactant selected from the group of;

Alkylglucamide comprising a saturated or unsaturated, branched, or unbranched C₆ to C₂₂, preferably C₁₀ to C₁₈, more preferably C₁₂ to C₁₆ alkyl group,

Alkyl fructoside comprising a saturated or unsaturated, branched, or unbranched C₆ to C₂₂, preferably C₁₀ to C₁₈, more preferably C₁₂ to C₁₆ alkyl group,

An alkyl glucoside comprising a saturated or unsaturated, branched, or unbranched C₆ to C₂₂, preferably C₁₀ to C₁₈, more preferably C₁₂ to C₁₆ alkyl group, and

Alkyl alcohol alkoxylate of the formula R₁₀(OR₁₁)ₘOH, in which R₁₀ represents a linear or branched C₆-C₂₂, preferably C₁₀-C₁₈, more preferably C₁₂-C₁₆ alkyl group, R₁₁ represents a C₂-C₄, preferably a C₂ alkyl group, and m represents 1 to about 10, preferably 2 to about 6, more preferably 2 to 6,

According to preferred embodiments of the present disclosure, one or more anionic surfactants are included in the cosmetic composition, which is preferably selected from the group of;

straight-chain or branched, saturated or mono- or polyunsaturated alkyl sulfonates containing from about 8 to about 24, preferably from about 12 to about 22, more preferably from about 16 to about 18 carbon atoms,

linear alpha-olefin sulfonates with from about 8 to about 24, preferably from about 12 to about 22, more preferably from about 16 to about 18 C atoms,

Alkyl sulfates and alkyl polyglycol ether sulfates of the formula R₉—O—(CH₂—CH₂O)ₙ—SO₃X, in which R₉ is preferably a straight-chain or branched, saturated or mono- or polyunsaturated alkyl or alkenyl radical having from about 8 to about 24, preferably from about 12 to about 22, more preferably from about 16 to about 18 carbon atoms, n is 0 or 1 to about 12, more preferably 2 to 4, and X is an alkali metal or alkaline earth metal ion or protonated triethanolamine or the ammonium ion,

straight-chain or branched, saturated or mono- or polyunsaturated alkylcarboxylic acids containing from about 8 to about 24, preferably from about 12 to about 22, more preferably from about 16 to about 18 carbon atoms,

straight-chain or branched, saturated or mono- or polyunsaturated alkyl phosphates containing from about 8 to about 24, preferably from about 12 to about 22, more preferably from about 16 to about 18 carbon atoms,

Alkyl isethionate whose alkyl group is selected from a branched or unbranched C₆ to about C₂₂, preferably from about C₁₀ to about C₁₈, more preferably from about C₁₂ to about C₁₆ alkyl group, in particular sodium cocoyl isethionate,

Alkyl glycoside carboxylic acids whose alkyl group is selected from a branched or unbranched C₆ to about C₂₂, preferably from about C₁₀ to about C₁₈, more preferably from about C₁₂ to about C₁₆ alkyl group,

Alkyl sulfosuccinates, the two alkyl groups of which are selected from identical or different, branched, or unbranched C₂ to about C₁₂, preferably C₄ to about C₁₀, more preferably from about C₆ to about C₈ alkyl groups,

Alkyl taurates, the alkyl group of which is selected from a branched or unbranched about C₆ to about C₂₂, preferably from about C₁₀ to about C₁₈, more preferably from about C₁₂ to about C₁₆ alkyl group,

Alkyl sarcosinates whose alkyl group is selected from a branched or unbranched about C₆ to about C₂₂, preferably from about C₁₀ to about C₁₈, more preferably from about C₁₂ to about C₁₆ alkyl group,

Sulfonates of unsaturated fatty acids with from about 8 to about 24, preferably from about 12 to about 22, more preferably from about 16 to about 18 C atoms and 1 to 6 double bonds, wherein the counterion of the anionic surfactant is an alkali or alkaline earth metal ion or a protonated triethanolamine or the ammonium ion.

Particularly preferred anionic surfactants are straight-chain or branched alkyl ether sulfates containing an alkyl radical with from about 8 to about 18 and with from about 10 to about 16 carbon atoms and 1 to 6 and 2 to 4 ethylene oxide units. Very preferably, the surfactant mixture of anionic and amphoteric/zwitterionic surfactants contains sodium lauryl ether sulfate (INCI: sodium laureth sulfate) and very preferably sodium lauryl ether sulfate with 2 ethylene oxide units.

Amphoteric surfactants, also known as zwitterionic surfactants, are surface-active compounds that contain at least one quaternary ammonium group and at least one —COO⁻ or —SO₃⁻ group in the molecule. Amphoteric/zwitterionic surfactants also include surface-active compounds which, in addition to a C₈-C₂₄ alkyl or acyl group, contain at least one free amino group and at least one —COOH— or —SO₃H— group and can form internal salts.

According to a preferred embodiment of the present disclosure, the amphoteric surfactants in the cosmetic composition are selected from the group of:

Alkyl betaine comprising at least one saturated or unsaturated, branched, or unbranched about C₆ to about C₂₂, preferably from about C₁₀ to about C₁₈, more preferably from about C₁₂ to about C₁₆ alkyl group,

Alkyl amphodiacetate or alkyl amphodiacetate comprising a saturated or unsaturated, branched, or unbranched about C₆ to about C₂₂, preferably from about C₁₀ to about C₁₈, more preferably from about C₁₂ to about C₁₆ alkyl group, with an alkali or alkaline earth metal counterion, and

Alkylamidopropyl betaine comprising at least one saturated or unsaturated, branched, or unbranched about C₆ to about C₂₂, preferably from about C₁₀ to about C₁₈, more preferably from about C₁₂ to about C₁₆ alkyl group.

Particularly suitable amphoteric/zwitterionic surfactants include those known under the INCI designation cocamidopropyl betaine and disodium cocoamphodiacetate.

According to a preferred embodiment of the present disclosure, the nonionic surfactant is selected from the group of:

Alkylglucamide comprising a saturated or unsaturated, branched, or unbranched about C₆ to about C₂₂, preferably from about C₁₀ to about C₁₈, more preferably from about C₁₂ to about C₁₆ alkyl group,

Alkyl fructoside comprising a saturated or unsaturated, branched, or unbranched about C₆ to about C₂₂, preferably from about C₁₀ to about C₁₈, more preferably from about C₁₂ to about C₁₆ alkyl group,

An alkyl glucoside comprising a saturated or unsaturated, branched, or unbranched about C₆ to about C₂₂, preferably from about C₁₀ to about C₁₈, more preferably from about C₁₂ to about C₁₆ alkyl group,

Alkyl alcohol alkoxylate of the formula R₁₀(OR₁₁)ₘOH, in which R₁₀ represents a linear or branched about C₆-about C₂₂, preferably from about C₁₀-about C₁₈, more preferably from about C₁₂-about C₁₆ alkyl group, R₁₁ represents a C₂-C₄, preferably a C₂ alkyl group, and m represents 1 to about 10, preferably 2 to about 6, more preferably 2 to 6, and

Alkyl esters of the formula R₁₂COOR₁₃, in which R₁₂ represents a linear or branched about C₆ to about C₂₂, preferably about C₁₀ to about C₁₈, more preferably about C₁₂ to about C₁₆ alkyl group, R₁₃ represents a C₁ to C₄, preferably a C₂ alkyl group.

According to a preferred embodiment of the present disclosure, the cosmetic composition contains two structurally different surfactants. It is particularly preferred that the cosmetic agent contains two structurally different surfactants from one another, preferably the cosmetic agent contains two structurally different cationic surfactants from one another, or the cosmetic agent contains a cationic surfactant and a nonionic surfactant.

The cosmetic composition may contain, in addition or as an alternative to a synthetic polymer, at least one natural or synthetic wax having a melting point above about 37° C. as a firming compound.

Natural or synthetic waxes can be solid kerosenes or isoparaffins, vegetable waxes such as candelilla wax, camauba wax, esparto grass wax, Japan wax, cork wax, sugar cane wax, ouricury wax, montan wax, sunflower wax, fruit waxes and animal waxes such as beeswaxes and other insect waxes, Whale wax, shellac wax, wool wax and brushing grease, furthermore mineral waxes, such as ceresin and ozokerite or petrochemical waxes, such as petrolatum, kerosene waxes, microwaxes of polyethylene or polypropylene and polyethylene glycol waxes can be used. It may be advantageous to use hydrogenated or cured waxes. Chemically modified waxes, in particular hard waxes such as montan ester waxes, sasol waxes and hydrogenated jojoba waxes, can also be used.

Furthermore, in addition to the mandatory components, the triglycerides of saturated and optionally hydroxylated about C16-30 fatty acids, such as hydrogenated triglyceride fats (hydrogenated palm oil, hydrogenated coconut oil, hydrogenated castor oil), glyceryl tribehenate or glyceryl tri-12-hydroxystearate are suitable in the cosmetic products.

The wax components can also be selected from the group of esters of saturated, unbranched alkanecarboxylic acids having a chain length of from about 22 to about 44 carbon atoms and saturated, unbranched alcohols having a chain length of from about 22 to about 44 carbon atoms, provided that the wax component or the totality of wax components are solid at room temperature. Silicone waxes, for example stearyltrimethylsilane/stearyl alcohol, may also be beneficial.

Natural, chemically modified, and synthetic waxes can be used alone or in combination. Thus, several waxes can also be used. Furthermore, several wax mixtures, possibly mixed with other additives, are also commercially available. The products sold under the designations “Special Wax 7686 OE” (a mixture of cetyl palmitate, beeswax, microcrystalline wax and polyethylene with a melting range of about 73-75° C.; manufacturer: Kahl® & Co), Polywax® GP 200 (a mixture of stearyl alcohol and polyethylene glycol stearate with a melting point of about 47-51° C.; manufacturer: Croda®) and “Softceresin® FL 400” (a vaseline/vaseline oil/wax mixture with a melting point of about 50-54° C.; manufacturer: Parafluid Mineral Oil Company) are examples of mixtures that can be used.

Preferably, the wax is selected from carnauba wax (INCI: Copernicia Cerifera Cera) Bienenwachs (INCI: Beeswax), petrolatum (INCI), microcrystalline wax and especially mixtures thereof.

Preferred blends include the combination of carnauba wax (INCI: Copernicia Cerifera Cera), petrolatum and microcrystalline wax or the combination of beeswax (INCI: Beeswax) and petrolatum.

The wax or wax components should be solid at about 25° C. and should melt in the range >about 37° C.

The composition for treating a keratinous material preferably contains the firming compound in a total amount of from about 0.5 to about 50% by weight, preferably from about 1 to about 40% by weight, more preferably from about 1.5 to about 30% by weight, even more preferably from about 2 to about 25% by weight, based on the total weight of the cosmetic composition.

Other suitable ingredients include nonionic polymers, anionic polymers, waxes, protein hydrolysates, amino acids, oligopetides, vitamins, provitamins, vitamin precursors, betaines, bioquinones, purine (derivatives), plant extracts, silicones, ester oils, UV light protection filters, structuring agents, thickeners, electrolytes, pH-adjusting agents, swelling agents, colorants, anti-dandruff agents, complexing agents, opacifiers, pearlescent agents, pigments, stabilizing agents, propellants, antioxidants, perfume oils and/or preservatives.

In preferred embodiments 1 to 188, the preferred organic silicon compounds are combined with the preferred cationic polymers in a cosmetic composition as contemplated herein.

| | | |
|---|---|---|
| 1 | 3-(Triethoxysilyl)-N-[3-(triethoxysilyl)propyl]-1- | Polyquaternium-1 |
| | propanamine | |
| 2 | 3-(Triethoxysilyl)-N-[3-(triethoxysilyl)propyl]-1- | Polyquaternium-2 |
| | propanamine | |
| 3 | 3-(Triethoxysilyl)-N-[3-(triethoxysilyl)propyl]-1- | Chitosan |
| | propanamine | |
| 4 | 3-(Triethoxysilyl)-N-[3-(triethoxysilyl)propyl]-1- | Polyquaternium-4 |
| | propanamine | |
| 5 | 3-(Triethoxysilyl)-N-[3-(triethoxysilyl)propyl]-1- | Polyquaternium-5 |
| | propanamine | |
| 6 | 3-(Triethoxysilyl)-N-[3-(triethoxysilyl)propyl]-1- | Polyquaternium-6 |
| | propanamine | |
| 7 | 3-(Triethoxysilyl)-N-[3-(triethoxysilyl)propyl]-1- | Polyquaternium-7 |
| | propanamine | |
| 8 | 3-(Triethoxysilyl)-N-[3-(triethoxysilyl)propyl]-1- | Polyquaternium-8 |
| | propanamine | |
| 9 | 3-(Triethoxysilyl)-N-[3-(triethoxysilyl)propyl]-1- | Polyquaternium-9 |
| | propanamine | |
| 10 | 3-(Triethoxysilyl)-N-[3-(triethoxysilyl)propyl]-1- | Polyquaternium-10 |
| | propanamine | |
| 11 | 3-(Triethoxysilyl)-N-[3-(triethoxysilyl)propyl]-1- | Polyquaternium-11 |
| | propanamine | |
| 12 | 3-(Triethoxysilyl)-N-[3-(triethoxysilyl)propyl]-1- | Polyquaternium-12 |
| | propanamine | |
| 13 | 3-(Triethoxysilyl)-N-[3-(triethoxysilyl)propyl]-1- | Polyquaternium-13 |
| | propanamine | |
| 14 | 3-(Triethoxysilyl)-N-[3-(triethoxysilyl)propyl]-1- | Polyquaternium-14 |
| | propanamine | |
| 15 | 3-(Triethoxysilyl)-N-[3-(triethoxysilyl)propyl]-1- | Polyquaternium-15 |
| | propanamine | |
| 16 | 3-(Triethoxysilyl)-N-[3-(triethoxysilyl)propyl]-1- | Polyquaternium-16 |
| | propanamine | |
| 17 | 3-(Triethoxysilyl)-N-[3-(triethoxysilyl)propyl]-1- | Polyquaternium-17 |
| | propanamine | |
| 18 | 3-(Triethoxysilyl)-N-[3-(triethoxysilyl)propyl]-1- | Polyquaternium-18 |
| | propanamine | |
| 19 | 3-(Triethoxysilyl)-N-[3-(triethoxysilyl)propyl]-1- | Polyquaternium-19 |
| | propanamine | |
| 20 | 3-(Triethoxysilyl)-N-[3-(triethoxysilyl)propyl]-1- | Polyquaternium-20 |
| | propanamine | |
| 21 | 3-(Triethoxysilyl)-N-[3-(triethoxysilyl)propyl]-1- | Polyquaternium-22 |
| | propanamine | |
| 22 | 3-(Triethoxysilyl)-N-[3-(triethoxysilyl)propyl]-1- | Polyquaternium-24 |
| | propanamine | |
| 23 | 3-(Triethoxysilyl)-N-[3-(triethoxysilyl)propyl]-1- | Polyquaternium-27 |
| | propanamine | |
| 24 | 3-(Triethoxysilyl)-N-[3-(triethoxysilyl)propyl]-1- | Polyquaternium-28 |
| | propanamine | |
| 25 | 3-(Triethoxysilyl)-N-[3-(triethoxysilyl)propyl]-1- | Polyquaternium-29 |
| | propanamine | |
| 26 | 3-(Triethoxysilyl)-N-[3-(triethoxysilyl)propyl]-1- | Polyquaternium-30 |
| | propanamine | |
| 27 | 3-(Triethoxysilyl)-N-[3-(triethoxysilyl)propyl]-1- | Polyquaternium-31 |
| | propanamine | |
| 28 | 3-(Triethoxysilyl)-N-[3-(triethoxysilyl)propyl]-1- | Polyquaternium-32 |
| | propanamine | |
| 29 | 3-(Triethoxysilyl)-N-[3-(triethoxysilyl)propyl]-1- | Polyquaternium-33 |
| | propanamine | |
| 30 | 3-(Triethoxysilyl)-N-[3-(triethoxysilyl)propyl]-1- | Polyquaternium-34 |
| | propanamine | |
| 31 | 3-(Triethoxysilyl)-N-[3-(triethoxysilyl)propyl]-1- | Polyquaternium-35 |
| | propanamine | |
| 32 | 3-(Triethoxysilyl)-N-[3-(triethoxysilyl)propyl]-1- | Polyquaternium-36 |
| | propanamine | |
| 33 | 3-(Triethoxysilyl)-N-[3-(triethoxysilyl)propyl]-1- | Polyquaternium-37 |
| | propanamine | |
| 34 | 3-(Triethoxysilyl)-N-[3-(triethoxysilyl)propyl]-1- | Polyquaternium-39 |
| | propanamine | |
| 35 | 3-(Triethoxysilyl)-N-[3-(triethoxysilyl)propyl]-1- | Polyquaternium-45 |
| | propanamine | |
| 36 | 3-(Triethoxysilyl)-N-[3-(triethoxysilyl)propyl]-1- | Polyquaternium-46 |
| | propanamine | |
| 37 | 3-(Triethoxysilyl)-N-[3-(triethoxysilyl)propyl]-1- | Polyquaternium-47 |
| | propanamine | |
| 38 | 3-(Triethoxysilyl)-N-[3-(triethoxysilyl)propyl]-1- | Polyquaternium-48 |
| | propanamine | |
| 39 | 3-(Triethoxysilyl)-N-[3-(triethoxysilyl)propyl]-1- | Polyquaternium-49 |
| | propanamine | |
| 40 | 3-(Triethoxysilyl)-N-[3-(triethoxysilyl)propyl]-1- | Polyquaternium-50 |
| | propanamine | |
| 41 | 3-(Triethoxysilyl)-N-[3-(triethoxysilyl)propyl]-1- | Polyquaternium-55 |
| | propanamine | |
| 42 | 3-(Triethoxysilyl)-N-[3-(triethoxysilyl)propyl]-1- | Polyquaternium-56 |
| | propanamine | |
| 43 | 3-(Triethoxysilyl)-N-[3-(triethoxysilyl)propyl]-1- | Polyquaternium-67 |
| | propanamine | |
| 44 | 3-(Triethoxysilyl)-N-[3-(triethoxysilyl)propyl]-1- | Polyquaternium-72 |
| | propanamine | |
| 45 | (3-Aminopropyl)triethoxysilane | Polyquaternium-1 |
| 46 | (3-Aminopropyl)triethoxysilane | Polyquaternium-2 |
| 47 | (3-Aminopropyl)triethoxysilane | Chitosan |
| 48 | (3-Aminopropyl)triethoxysilane | Polyquaternium-4 |
| 49 | (3-Aminopropyl)triethoxysilane | Polyquaternium-5 |
| 50 | (3-Aminopropyl)triethoxysilane | Polyquaternium-6 |
| 51 | (3-Aminopropyl)triethoxysilane | Polyquaternium-7 |
| 52 | (3-Aminopropyl)triethoxysilane | Polyquaternium-8 |
| 53 | (3-Aminopropyl)triethoxysilane | Polyquaternium-9 |
| 54 | (3-Aminopropyl)triethoxysilane | Polyquaternium-10 |
| 55 | (3-Aminopropyl)triethoxysilane | Polyquaternium-11 |
| 56 | (3-Aminopropyl)triethoxysilane | Polyquaternium-12 |
| 57 | (3-Aminopropyl)triethoxysilane | Polyquaternium-13 |
| 58 | (3-Aminopropyl)triethoxysilane | Polyquaternium-14 |
| 59 | (3-Aminopropyl)triethoxysilane | Polyquaternium-15 |
| 60 | (3-Aminopropyl)triethoxysilane | Polyquaternium-16 |
| 61 | (3-Aminopropyl)triethoxysilane | Polyquaternium-17 |
| 62 | (3-Aminopropyl)triethoxysilane | Polyquaternium-18 |
| 63 | (3-Aminopropyl)triethoxysilane | Polyquaternium-19 |
| 64 | (3-Aminopropyl)triethoxysilane | Polyquaternium-20 |
| 65 | (3-Aminopropyl)triethoxysilane | Polyquaternium-22 |
| 66 | (3-Aminopropyl)triethoxysilane | Polyquaternium-24 |
| 67 | (3-Aminopropyl)triethoxysilane | Polyquaternium-27 |
| 68 | (3-Aminopropyl)triethoxysilane | Polyquaternium-28 |
| 69 | (3-Aminopropyl)triethoxysilane | Polyquaternium-29 |
| 70 | (3-Aminopropyl)triethoxysilane | Polyquaternium-30 |
| 71 | (3-Aminopropyl)triethoxysilane | Polyquaternium-31 |
| 72 | (3-Aminopropyl)triethoxysilane | Polyquaternium-32 |
| 73 | (3-Aminopropyl)triethoxysilane | Polyquaternium-33 |
| 74 | (3-Aminopropyl)triethoxysilane | Polyquaternium-34 |
| 75 | (3-Aminopropyl)triethoxysilane | Polyquaternium-35 |
| 76 | (3-Aminopropyl)triethoxysilane | Polyquaternium-36 |
| 77 | (3-Aminopropyl)triethoxysilane | Polyquaternium-37 |
| 78 | (3-Aminopropyl)triethoxysilane | Polyquaternium-39 |
| 79 | (3-Aminopropyl)triethoxysilane | Polyquaternium-45 |
| 80 | (3-Aminopropyl)triethoxysilane | Polyquaternium-46 |
| 81 | (3-Aminopropyl)triethoxysilane | Polyquaternium-47 |
| 82 | (3-Aminopropyl)triethoxysilane | Polyquaternium-48 |
| 83 | (3-Aminopropyl)triethoxysilane | Polyquaternium-49 |
| 84 | (3-Aminopropyl)triethoxysilane | Polyquaternium-50 |
| 85 | (3-Aminopropyl)triethoxysilane | Polyquaternium-55 |
| 86 | (3-Aminopropyl)triethoxysilane | Polyquaternium-56 |
| 87 | (3-Aminopropyl)triethoxysilane | Polyquaternium-67 |
| 88 | (3-Aminopropyl)triethoxysilane | Polyquaternium-72 |
| 89 | (3-Aminopropyl)triethoxysilane | AMP- |
| | | Acrylates/Dimethylaminoethyl- |
| | | methacrylate Copolymer |
| 90 | 3-(Triethoxysilyl)-N-[3-(triethoxysilyl)propyl]-1- | AMP- |
| | propanamine | Acrylates/Dimethylaminoethyl |
| | | methacrylate Copolymer |
| 91 | (3-Aminopropyl)triethoxysilane + | Polyquaternium-1 |
| | methyltrimethoxysilane | |
| 92 | (3-Aminopropyl)triethoxysilane + | Polyquaternium-1 |
| | methyltriethoxysilane | |
| 93 | (3-Aminopropyl)triethoxysilane + | Polyquaternium-2 |
| | methyltrimethoxysilane | |
| 94 | (3-Aminopropyl)triethoxysilane + | Polyquaternium-2 |
| | methyltriethoxysilane | |
| 95 | (3-Aminopropyl)triethoxysilane + | Chitosan |
| | methyltrimethoxysilane | |
| 96 | (3-Aminopropyl)triethoxysilane + | Chitosan |
| | methyltriethoxysilane | |
| 97 | (3-Aminopropyl)triethoxysilane + | Polyquaternium-4 |
| | methyltrimethoxysilane | |
| 98 | (3-Aminopropyl)triethoxysilane + | Polyquaternium-4 |
| | methyltriethoxysilane | |
| 99 | (3-Aminopropyl)triethoxysilane + | Polyquaternium-5 |
| | methyltrimethoxysilane | |
| 100 | (3-Aminopropyl)triethoxysilane + | Polyquaternium-5 |
| | methyltriethoxysilane | |
| 101 | (3-Aminopropyl)triethoxysilane + | Polyquaternium-6 |
| | methyltrimethoxysilane | |
| 102 | (3-Aminopropyl)triethoxysilane + | Polyquaternium-6 |
| | methyltriethoxysilane | |
| 103 | (3-Aminopropyl)triethoxysilane + | Polyquaternium-7 |
| | methyltrimethoxysilane | |
| 104 | (3-Aminopropyl)triethoxysilane + | Polyquaternium-7 |
| | methyltriethoxysilane | |
| 105 | (3-Aminopropyl)triethoxysilane + | Polyquaternium-8 |
| | methyltrimethoxysilane | |
| 106 | (3-Aminopropyl)triethoxysilane + | Polyquaternium-8 |
| | methyltriethoxysilane | |
| 107 | (3-Aminopropyl)triethoxysilane + | Polyquaternium-9 |
| | methyltrimethoxysilane | |
| 108 | (3-Aminopropyl)triethoxysilane + | Polyquaternium-9 |
| | methyltriethoxysilane | |
| 109 | (3-Aminopropyl)triethoxysilane + | Polyquaternium-10 |
| | methyltrimethoxysilane | |
| 110 | (3-Aminopropyl)triethoxysilane + | Polyquaternium-10 |
| | methyltriethoxysilane | |
| 111 | (3-Aminopropyl)triethoxysilane + | Polyquaternium-11 |
| | methyltrimethoxysilane | |
| 112 | (3-Aminopropyl)triethoxysilane + | Polyquaternium-11 |
| | methyltriethoxysilane | |
| 113 | (3-Aminopropyl)triethoxysilane + | Polyquaternium-12 |
| | methyltrimethoxysilane | |
| 114 | (3-Aminopropyl)triethoxysilane + | Polyquaternium-12 |
| | methyltriethoxysilane | |
| 115 | (3-Aminopropyl)triethoxysilane + | Polyquaternium-13 |
| | methyltrimethoxysilane | |
| 116 | (3-Aminopropyl)triethoxysilane + | Polyquaternium-13 |
| | methyltriethoxysilane | |
| 117 | (3-Aminopropyl)triethoxysilane + | Polyquaternium-14 |
| | methyltrimethoxysilane | |
| 118 | (3-Aminopropyl)triethoxysilane + | Polyquaternium-14 |
| | methyltriethoxysilane | |
| 119 | (3-Aminopropyl)triethoxysilane + | Polyquaternium-15 |
| | methyltrimethoxysilane | |
| 120 | (3-Aminopropyl)triethoxysilane + | Polyquaternium-15 |
| | methyltriethoxysilane | |
| 121 | (3-Aminopropyl)triethoxysilane + | Polyquaternium-16 |
| | methyltrimethoxysilane | |
| 122 | (3-Aminopropyl)triethoxysilane + | Polyquaternium-16 |
| | methyltriethoxysilane | |
| 123 | (3-Aminopropyl)triethoxysilane + | Polyquaternium-17 |
| | methyltrimethoxysilane | |
| 124 | (3-Aminopropyl)triethoxysilane + | Polyquaternium-17 |
| | methyltriethoxysilane | |
| 125 | (3-Aminopropyl)triethoxysilane + | Polyquaternium-18 |
| | methyltrimethoxysilane | |
| 126 | (3-Aminopropyl)triethoxysilane + | Polyquaternium-18 |
| | methyltriethoxysilane | |
| 127 | (3-Aminopropyl)triethoxysilane + | Polyquaternium-19 |
| | methyltrimethoxysilane | |
| 128 | (3-Aminopropyl)triethoxysilane + | Polyquaternium-19 |
| | methyltriethoxysilane | |
| 129 | (3-Aminopropyl)triethoxysilane + | Polyquaternium-20 |
| | methyltrimethoxysilane | |
| 130 | (3-Aminopropyl)triethoxysilane + | Polyquaternium-20 |
| | methyltriethoxysilane | |
| 131 | (3-Aminopropyl)triethoxysilane + | Polyquaternium-21 |
| | methyltrimethoxysilane | |
| 132 | (3-Aminopropyl)triethoxysilane + | Polyquaternium-21 |
| | methyltriethoxysilane | |
| 133 | (3-Aminopropyl)triethoxysilane + | Polyquaternium-22 |
| | methyltrimethoxysilane | |
| 134 | (3-Aminopropyl)triethoxysilane + | Polyquaternium-22 |
| | methyltriethoxysilane | |
| 135 | (3-Aminopropyl)triethoxysilane + | Polyquaternium-23 |
| | methyltrimethoxysilane | |
| 136 | (3-Aminopropyl)triethoxysilane + | Polyquaternium-23 |
| | methyltriethoxysilane | |
| 137 | (3-Aminopropyl)triethoxysilane + | Polyquaternium-24 |
| | methyltrimethoxysilane | |
| 138 | (3-Aminopropyl)triethoxysilane + | Polyquaternium-24 |
| | methyltriethoxysilane | |
| 139 | (3-Aminopropyl)triethoxysilane + | Polyquaternium-25 |
| | methyltrimethoxysilane | |
| 140 | (3-Aminopropyl)triethoxysilane + | Polyquaternium-25 |
| | methyltriethoxysilane | |
| 141 | (3-Aminopropyl)triethoxysilane + | Polyquaternium-26 |
| | methyltrimethoxysilane | |
| 142 | (3-Aminopropyl)triethoxysilane + | Polyquaternium-26 |
| | methyltriethoxysilane | |
| 143 | (3-Aminopropyl)triethoxysilane + | Polyquaternium-27 |
| | methyltrimethoxysilane | |
| 144 | (3-Aminopropyl)triethoxysilane + | Polyquaternium-27 |
| | methyltriethoxysilane | |
| 145 | (3-Aminopropyl)triethoxysilane + | Polyquaternium-28 |
| | methyltrimethoxysilane | |
| 146 | (3-Aminopropyl)triethoxysilane + | Polyquaternium-28 |
| | methyltriethoxysilane | |
| 147 | (3-Aminopropyl)triethoxysilane + | Polyquaternium-29 |
| | methyltrimethoxysilane | |
| 148 | (3-Aminopropyl)triethoxysilane + | Polyquaternium-29 |
| | methyltriethoxysilane | |
| 149 | (3-Aminopropyl)triethoxysilane + | Polyquaternium-30 |
| | methyltrimethoxysilane | |
| 150 | (3-Aminopropyl)triethoxysilane + | Polyquaternium-30 |
| | methyltriethoxysilane | |
| 151 | (3-Aminopropyl)triethoxysilane + | Polyquaternium-31 |
| | methyltrimethoxysilane | |
| 152 | (3-Aminopropyl)triethoxysilane + | Polyquaternium-31 |
| | methyltriethoxysilane | |
| 153 | (3-Aminopropyl)triethoxysilane + | Polyquaternium-32 |
| | methyltrimethoxysilane | |
| 154 | (3-Aminopropyl)triethoxysilane + | Polyquaternium-32 |
| | methyltriethoxysilane | |
| 155 | (3-Aminopropyl)triethoxysilane + | Polyquaternium-33 |
| | methyltrimethoxysilane | |
| 156 | (3-Aminopropyl)triethoxysilane + | Polyquaternium-33 |
| | methyltriethoxysilane | |
| 157 | (3-Aminopropyl)triethoxysilane + | Polyquaternium-34 |
| | methyltrimethoxysilane | |
| 158 | (3-Aminopropyl)triethoxysilane + | Polyquaternium-34 |
| | methyltriethoxysilane | |
| 159 | (3-Aminopropyl)triethoxysilane + | Polyquaternium-35 |
| | methyltrimethoxysilane | |
| 160 | (3-Aminopropyl)triethoxysilane + | Polyquaternium-35 |
| | methyltriethoxysilane | |
| 161 | (3-Aminopropyl)triethoxysilane + | Polyquaternium-36 |
| | methyltrimethoxysilane | |
| 162 | (3-Aminopropyl)triethoxysilane + | Polyquaternium-36 |
| | methyltriethoxysilane | |
| 163 | (3-Aminopropyl)triethoxysilane + | Polyquaternium-37 |
| | methyltrimethoxysilane | |
| 164 | (3-Aminopropyl)triethoxysilane + | Polyquaternium-37 |
| | methyltriethoxysilane | |
| 165 | (3-Aminopropyl)triethoxysilane + | Polyquaternium-38 |
| | methyltrimethoxysilane | |
| 166 | (3-Aminopropyl)triethoxysilane + | Polyquaternium-38 |
| | methyltriethoxysilane | |
| 167 | (3-Aminopropyl)triethoxysilane + | Polyquaternium-39 |
| | methyltrimethoxysilane | |
| 168 | (3-Aminopropyl)triethoxysilane + | Polyquaternium-39 |
| | methyltriethoxysilane | |
| 169 | (3-Aminopropyl)triethoxysilane + | Polyquaternium-45 |
| | methyltrimethoxysilane | |
| 170 | (3-Aminopropyl)triethoxysilane + | Polyquaternium-45 |
| | methyltriethoxysilane | |
| 171 | (3-Aminopropyl)triethoxysilane + | Polyquaternium-46 |
| | methyltrimethoxysilane | |
| 172 | (3-Aminopropyl)triethoxysilane + | Polyquaternium-46 |
| | methyltriethoxysilane | |
| 173 | (3-Aminopropyl)triethoxysilane + | Polyquaternium-47 |
| | methyltrimethoxysilane | |
| 174 | (3-Aminopropyl)triethoxysilane + | Polyquaternium-47 |
| | methyltriethoxysilane | |
| 175 | (3-Aminopropyl)triethoxysilane + | Polyquaternium-48 |
| | methyltrimethoxysilane | |
| 176 | (3-Aminopropyl)triethoxysilane + | Polyquaternium-48 |
| | methyltriethoxysilane | |
| 177 | (3-Aminopropyl)triethoxysilane + | Polyquaternium-50 |
| | methyltrimethoxysilane | |
| 178 | (3-Aminopropyl)triethoxysilane + | Polyquaternium-50 |
| | methyltriethoxysilane | |
| 179 | (3-Aminopropyl)triethoxysilane + | Polyquaternium-55 |
| | methyltrimethoxysilane | |
| 180 | (3-Aminopropyl)triethoxysilane + | Polyquaternium-55 |
| | methyltriethoxysilane | |
| 181 | (3-Aminopropyl)triethoxysilane + | Polyquaternium-56 |
| | methyltrimethoxysilane | |
| 182 | (3-Aminopropyl)triethoxysilane + | Polyquaternium-56 |
| | methyltriethoxysilane | |
| 183 | (3-Aminopropyl)triethoxysilane + | Polyquaternium-67 |
| | methyltrimethoxysilane | |
| 184 | (3-Aminopropyl)triethoxysilane + | Polyquaternium-67 |
| | methyltriethoxysilane | |
| 185 | (3-Aminopropyl)triethoxysilane + | Polyquaternium-72 |
| | methyltrimethoxysilane | |
| 186 | (3-Aminopropyl)triethoxysilane + | Polyquaternium-72 |
| | methyltriethoxysilane | |
| 187 | (3-Aminopropyl)triethoxysilane + | AMP- |
| | methyltrimethoxysilane | Acrylates/Dimethylaminoethyl- |
| | | methacrylate Copolymer |
| 188 | (3-Aminopropyl)triethoxysilane + | AMP- |
| | methyltriethoxysilane | Acrylates/Dimethylaminoethyl |
| | | methacrylate Copolymer |

The combinations in the above table represent active ingredient combinations that are combined in cosmetic products with other components described above.

The active ingredient combination of at least one organic silicon compound and the cationic polymer may already be present in the agent for treating a keratinous material. In this embodiment, the agent for treating a keratinous material is already distributed in a form ready for use. To provide a formulation that is as stable as possible during storage, the agent itself is preferably packaged with low or no water.

Alternatively, the at least one organic silicon compound is added a maximum of about 12 hours, preferably a maximum of about 6 hours, more preferably a maximum of about 3 hours, even more preferably a maximum of about 1 hour prior to application of the keratinous material treatment composition to a base comprising all the ingredients of the keratinous material treatment composition except the at least one organic silicon compound.

Furthermore, alternatively, the organic silicon compound and another component b) are added to a cosmetic product only shortly before use, i.e., from about 1 minute to about 12 hours, preferably from about 2 minutes to about 6 hours, particularly preferably from about 1 minute to about 3 hours, especially preferably from about 1 minute to about 1 hour.

In a further alternative, the AMEO or the bis(triethoxysilylpropyl)amine is added to an aqueous solution which is applied to the hair and, in a second step, an aqueous solution or a cosmetic composition containing the further component b) is applied to the hair.

For example, the user may first mix or shake an agent (α) comprising the organic silicon compound(s) with an agent (β) comprising the remaining ingredients of the agent for treating a keratinous material. The user can now apply this mixture of (α) and (β)—either directly after its preparation or after a short reaction time of from about 1 minute to about 20 minutes—to the keratinous materials. The agent (β) may contain water, in particular water in an amount >about 30% by weight, based on the total weight of the agent for treating keratinous materials.

Another object of the present application is the use of a cosmetic agent as contemplated herein for treating a keratinous material for the care of keratinous material and/or for hydrophobizing the surface of keratinous material.

About further preferred embodiments of use, the same applies mutatis mutandis as to the cosmetic agents.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for treating a keratinous material comprising
a) at least one organic silicon compound and
b) at least one cationic polymer.

2. The cosmetic composition for treating a keratinous material according to claim 1,
wherein:
the at least one organic silicon compound comprises a compound of the formula (I) and/or (II),
wherein in the organic silicon compound of formula (I)
R₁R₂N-L-Si(OR₃)ₐ(R₄)_{b} (I),
R₁, R₂ both represent a hydrogen atom,
L represents a linear, divalent C₁-C₆-alkylene group,
a stands for the number 3 and
b stands for the number 0, and
wherein in the organic silicon compound of formula (II)
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
R₅, R_{5′}, R_{5″}, R₆, R_{6′} and R_{6″} independently represent a C₁-C₆ alkyl group,
A, A′, A″, A′″ and A″″ independently represent a linear or branched C₁-C₂₀ divalent alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)
-(A″″)-Si(R₆″)_{d″}(OR₅″)_{c″} (III),
c stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
with the proviso that at least one of the residues from e, f, g, and h is different from 0.

3. The cosmetic composition for treating a keratinous material according to claim 2,
wherein
the agent for treating a keratinous material comprises at least one organic silicon compound of formula (I) selected from the group of
(3-Aminopropyl)trimethoxysilane
(3-Aminopropyl)triethoxysilane
(2-Aminoethyl)trimethoxysilane
(2-Aminoethyl)triethoxysilane
(3-Dimethylaminopropyl)trimethoxysilane
(3-Dimethylaminopropyl)triethoxysilane
(2-Dimethylaminoethyl)trimethoxysilane and
(2-Dimethylaminoethyl)triethoxysilane,
and/or wherein,
the agent for treating a keratinous material comprises at least one organic silicon compound of formula (II) selected from the group of
3-(trimethoxysilyl)-N-[3-(trimethoxysilyl)propyl]-1-propanamine
3-(triethoxysilyl)-N-[3-(triethoxysilyl)propyl]-1-propanamine
N-methyl-3-(trimethoxysilyl)-N-[3-(trimethoxysilyl)propyl]-1-propanamine
N-methyl-3-(triethoxysilyl)-N-[3-(triethoxysilyl)propyl]-1-propanamine
2-[Bis[3-(trimethoxysilyl)propyl]amino]-ethanol
2-[Bis[3-(triethoxysilyl)propyl]amino]ethanol
3-(trimethoxysilyl)-N,N-bis[3-(trimethoxysilyl)propyl]-1-propanamine
3-(triethoxysilyl)-N,N-bis[3-(triethoxysilyl)propyl]-1-propanamine
N1,N1-Bis[3-(trimethoxysilyl)propyl]-1,2-ethanediamine,
N1,N1-Bis[3-(triethoxysilyl)propyl]-1,2-ethanediamine,
N,N-Bis[3-(trimethoxysilyl)propyl]-2-propen-1-amine and
N,N-Bis[3-(triethoxysilyl)propyl]-2-propen-1-amine.

4. The cosmetic composition for treating a keratinous material according to claim 2, wherein the organic silicon compound of formula (I) is present in the cosmetic composition in an amount of from about 0.01 to about 10% by weight, based on a total weight of the cosmetic composition,
and wherein the organic silicon compound of formula (I) is 3-(triethoxysilyl)-N-[3-(triethoxysilyl)propyl]-1-propanamine.

5. The cosmetic composition for treating a keratinous material according to claim 1, wherein the at least one organic silicon comprises at least one organic silicon compound of formula (IV),
R₉Si(OR₁₀)ₖ(R₁₁)ₘ (IV),
which is selected from the group of
Methyltrimethoxysilane
Methyltriethoxysilane
Ethyltrimethoxysilane
Ethyltriethoxysilane
Propyltrimethoxysilane
Propyltriethoxysilane
Hexyltrimethoxysilane
Hexyltriethoxysilane
Octyltrimethoxysilane
Octyltriethoxysilane
Dodecyltrimethoxysilane
Dodecyltriethoxysilane
Octadecyltrimethoxysilane and
Octadecyltriethoxysilane.

6. The cosmetic composition for treating a keratinous material according to claim 1, wherein the cationic polymer is composed of monomer units having a cationic formal charge, wherein the amount of monomer units having a cationic formal charge exceeds the amount of monomer units having a formal charge other than the cationic formal charge.

7. The cosmetic composition for treating a keratinous material according to claim 1, wherein the cationic polymer is present in the cosmetic composition in an amount of from about 0.1 to about 10% by weight, based on a total weight of the cosmetic composition.

8. The cosmetic composition for treating a keratinous material according to claim 1, wherein the cosmetic composition further comprises a component c) which is a skin moisturizing agent selected from the group of glycerol, urea, hyaluronic acid, silanol esters of hyaluronic acid, panthenol, taurine, ceramides, phytosterols, aloe vera extracts, creatine, creatinine, sodium hyaluronate, polysaccharides, bio saccharides gum-1, cucumber extracts, butylene glycol, propylene glycol, methyl propanediol, ethylhexyl glycerin, sorbitol, amino acids, glycine, glycine soy, histidine, tyrosine, tryptophan, amino acid derivatives, lactic acid, lactates, and combinations thereof.

9. The cosmetic composition for treating a keratinous material according to claim 1, wherein the cosmetic composition for treating a keratinous material comprises at least two organic silicon compounds which are structurally different from one another.

10. The cosmetic composition for treating a keratinous material according to claim 1, wherein the composition for treating a keratinous material comprises, based on a total weight of the composition for treating a keratinous material:
from about 0.5 to about 3% by weight of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane, (2-dimethylaminoethyl)trimethoxysilane, and (2-dimethylaminoethyl)triethoxysilane, and
from about 3.2 to about 7% by weight of at least one second organic silicon compound selected from the group of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane, and octadecyltriethoxysilane.

11. A method for treating keratinous material, the method comprising the steps of:
Applying a cosmetic composition to the keratinous material, where the cosmetic composition comprises:
a) an organic silicon compound and
b) a cationic polymer.

12. The cosmetic composition for treating a keratinous material according to claim 2, wherein the organic silicon compound of formula (II) is present in an amount of from about 0.01 to about 10% by weight, based on a total weight of the cosmetic composition, and wherein the organic silicon compound of formula (II) is (3-aminopropyl)triethoxysilane.

13. The cosmetic composition for treating a keratinous material according to claim 1, wherein the cationic polymer is selected from the group of:
acrylamidopropyltrimonium chloride/acrylamide copolymer, acrylamidopropyltrimonium chloride/acrylates copolymer, acrylates/octylacrylamide/diphenyl amodimethicone copolymer, adipic acid/diethylenetriamine copolymer, adipic acid/dimethylaminohydroxypropyl diethylenetriamine copolymer, adipic acid/epoxypropyl diethylenetriamine copolymer, adipic acid/isophthalic acid/neopentyl glycol/trimethylolpropane copolymer, aminoethylpropanediol-acrylates/acrylamide copolymer, diethylene glycolamine/epichlorohydrin/piperazine copolymer, polyacrylamide, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-35, polyquaternium-36, polyquaternium-37, polyquaternium-39, polyquaternium-45, polyquaternium-46, polyquaternium-47, polyquaternium-48, polyquaternium-49, polyquaternium-50, polyquaternium-55, polyquaternium-56, polyquaternium-67, polyquaternium-72, vinylamine/vinyl alcohol copolymer, quaternized polyvinyl alcohol, cationic alkyl polyglycosides, chitosan, and vinyl caprolactam/VP/dimethylaminoethyl methacrylate copolymer.

14. The cosmetic composition for treating a keratinous material according to claim 1, wherein the cationic polymer is chitosan.

15. The cosmetic composition of claim 4 further comprising:
at least one organic silicon compound of formula (IV),
R₉Si(OR₁₀)ₖ(R₁₁)ₘ (IV),
selected from the group of;
methyltrimethoxysilane,
methyltriethoxysilane,
ethyltrimethoxysilane,
ethyltriethoxysilane,
propyltrimethoxysilane,
propyltriethoxysilane,
hexyltrimethoxysilane,
hexyltriethoxysilane,
octyltrimethoxysilane,
octyltriethoxysilane,
dodecyltrimethoxysilane,
dodecyltriethoxysilane,
octadecyltrimethoxysilane, and
octadecyltriethoxysilane.

16. The cosmetic composition of claim 12 further comprising:
at least one organic silicon compound of formula (IV),
R₉Si(OR₁₀)ₖ(R₁₁)ₘ (IV),
selected from the group of;
methyltrimethoxysilane,
methyltriethoxysilane,
ethyltrimethoxysilane,
ethyltriethoxysilane,
propyltrimethoxysilane,
propyltriethoxysilane,
hexyltrimethoxysilane,
hexyltriethoxysilane,
octyltrimethoxysilane,
octyltriethoxysilane,
dodecyltrimethoxysilane,
dodecyltriethoxysilane,
octadecyltrimethoxysilane, and
octadecyltriethoxysilane.

17. The cosmetic composition of claim 2, wherein the organic silicon compound of formula (I) is present in the cosmetic composition in an amount of from about 0.1 to about 4% by weight, based on a total weight of the cosmetic composition, and wherein the organic silicon compound of formula (I) is 3-(triethoxysilyl)-N-[3-(triethoxysilyl)propyl]-1-propanamine.

18. The cosmetic composition for treating a keratinous material according to claim 2, wherein the organic silicon compound of formula (II) is present in an amount of from about 0.1 to about 6% by weight, based on a total weight of the cosmetic composition, and wherein the organic silicon compound of formula (II) is (3-aminopropyl)triethoxysilane.

19. The cosmetic composition for treating a keratinous material according to claim 1, wherein the cationic polymer is present in the cosmetic composition in an amount of from about 1 to about 6% by weight, based on a total weight of the cosmetic composition.

20. The cosmetic composition of claim 10 further comprising:
a third organic silicon compound of formula (IV),
R₉Si(OR₁₀)ₖ(R₁₁)ₘ (IV),
wherein the third organic silicon compound is selected from the group of;
methyltrimethoxysilane,
methyltriethoxysilane,
ethyltrimethoxysilane,
ethyltriethoxysilane,
propyltrimethoxysilane,
propyltriethoxysilane,
hexyltrimethoxysilane,
hexyltriethoxysilane,
octyltrimethoxysilane,
octyltriethoxysilane,
dodecyltrimethoxysilane,
dodecyltriethoxysilane,
octadecyltrimethoxysilane, and
octadecyltriethoxysilane.
